(19) United States
(12) Patent Application Publication
(10) Pub. No.: US 2009/0177219 A1
(43) Pub. Date: Jul. 9, 2009

(54) FLEXIBLE TISSUE-PENETRATION INSTRUMENT WITH BLUNT TIP ASSEMBLY AND METHODS FOR PENETRATING TISSUE

(76) Inventor: Sean P. Conlon, Loveland, OH (US)

Correspondence Address: K&L GATES LLP, 535 SMITHFIELD STREET, PITTSBURGH, PA, 15222, US

(21) Appl. No.: 11/968,810

(22) Filed: Jan. 3, 2008

(51) Int. Cl. A61B 17/34; B65D 69/00; A61L 2/00

(52) U.S. Cl. 606/185; 206/363; 422/28

(57) ABSTRACT

A tissue-penetrating instrument. In various exemplary embodiments, the instrument may comprise a substantially flexible elongate penetration member that has a proximal end and a distal end. The distal end may have a substantially blunt tissue penetrating tip thereon. The instrument may further include an elongate hollow support tube that has a passage extending therethrough that is sized to movably support a portion of the substantially flexible elongate member therein and prevent buckling thereof as at least one motion is applied to the distal end of the substantially flexible elongate member. The elongate hollow support tube may be sized to extend through a working channel of an endoscope. Methods of using such instruments to perform surgical procedures by inserting the instrument through a natural body lumen are also disclosed.

### FIELD OF THE INVENTION

The present invention relates, in general, to surgical devices and methods of use and, more particularly, to devices and methods for forming openings through tissue walls during natural orifice transendoscopic surgical procedures.

### BACKGROUND OF THE INVENTION

Access to the abdominal cavity may, from time to time, be required for diagnostic and therapeutic endeavors for a variety of medical and surgical diseases. Historically, abdominal access has required a formal laparotomy to provide adequate access. Such procedures which require incisions to be made in the abdomen are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are commonly performed with an endoscope (including laparoscopes). Such procedures permit a physician to position, manipulate, and view medical instruments and accessories inside the patient through a small access opening in the patient's body. In this type of procedure, accessory devices are often inserted into a patient through trocars placed through the body wall.

Another way to access the abdominal cavity, however, is via natural openings (mouth, anus, vagina, urethra) of the body and through the peritoneal lining of the abdominal cavity. Such surgical approaches are generally known in the art as Natural Orifice Translumenal Endoscopic Surgery (NOTES)™ procedures. Obviously, the size and shape of instruments that may be passed through a bodily lumen in order to perform a medical procedure in the abdominal cavity are greatly restricted due to the anatomical properties of the lumen.

General surgeons, gastroenterologists, and other medical specialists routinely use flexible endoscopes for intraluminal (within the lumen of the alimentary canal) examination and treatment of the upper gastrointestinal (GI) tract, via the mouth, and the lower GI tract, via the anus. In these procedures, the physician pushes the flexible endoscope into the lumen, periodically pausing to articulate the distal end of the endoscope using external control knobs, to redirect the distal tip of the endoscope. In this way, the physician may navigate the crooked passageway of the upper GI past the pharynx, through the esophagus and gastro esophageal junction, and into the stomach. The physician must take great care not to injure the delicate mucosal lining of the lumen, which generally may stretch open to a diameter in the range of about 15-25 mm, but normally has a non-circular cross sectional configuration when relaxed.

During such translumenal procedures, a puncture must be formed in the stomach wall or in the gastrointestinal tract to access the peritoneal cavity. One device often used to form such a puncture is a needle knife which is inserted through the working channel of the endoscope, and which utilizes energy to penetrate through the tissue. A guide wire is then fed through the endoscope and is passed through the puncture in the stomach wall and into the peritoneal cavity. The needle knife is removed, leaving the guide wire as a placeholder. A balloon catheter is then passed over the guide wire and through the working channel of the endoscope to position the balloon within the opening in the stomach wall. The balloon can then be inflated to increase the size of the opening, thereby enabling the endoscope to push against the rear of the balloon and to be feed through the opening and into the peritoneal cavity. Once the endoscope is positioned within the peritoneal cavity, numerous procedures can be performed through the working channel of the endoscope.

While the current methods and devices used to penetrate tissue are effective, one drawback is the risk of damaging adjacent organs and tissue. Due to the low amount of energy and force of penetration needed to pass through tissue, there is the risk of penetrating adjacent tissue that is intended to be left unharmed during the procedure. Accordingly, there remains a need for improved tissue penetrating devices that can be used in connection with flexible endoscopes and reduce the likelihood of damaging non-target tissue. There also remains a need for a simplified procedure that requires less steps to form a puncture in tissue.

The foregoing discussion is intended only to illustrate some of the shortcomings present in the field of the invention at the time, and should not be taken as a disavowal of claim scope.

### SUMMARY

In one aspect of the invention, there is provided a tissue-penetration instrument. In various exemplary embodiments, the tissue-penetrating instrument may comprise a substantially flexible elongate penetration member that has a proximal end and a distal end. The distal end may have a substantially blunt tissue penetrating tip thereon. The instrument may further include an elongate hollow support tube that has a passage extending therethrough that is sized to movably support a portion of the substantially flexible elongate member therein and prevent buckling thereof as at least one motion is applied to the distal end of the substantially flexible elongate member. The elongate hollow support tube may be sized to extend through a working channel of an endoscope.

In another general aspect of various embodiments of the present invention, there is provided a tissue-penetration instrument that comprises a substantially flexible elongate penetration member that has a proximal end and a distal end. A control knob may be provided on the proximal end of the substantially flexible elongate penetration member to enable axial and rotational motions to be applied thereto. A blunt tissue-penetrating tip assembly may be coupled to the distal end of the substantially flexible elongate penetration member. The blunt tissue-penetrating tip assembly may have a substantially blunt end and at least one lateral time formed thereon. An elongate hollow support tube that has a passage extending therethrough sized to movably support a portion of the substantially flexible elongate member therein and prevent buckling thereof as at least one of the axial and rotational motions is applied to the control knob.

In still another general aspect of various embodiments of the present invention, there is provided a method for penetrating tissue. In various embodiments, the method may comprise inserting a substantially flexible dissection device through a body lumen and piercing partially through a portion of tissue with the dissection device to thereby form a partial penetration in the tissue. The method may further comprise inserting a substantially flexible tissue-penetration member that has a substantially blunt tissue penetrating tip through the body lumen such that the blunt tissue penetrating tip is positioned within the partial penetration formed in the tissue. In addition, the method may comprise applying at least one motion to the tissue-penetration member to cause the blunt tissue penetration tip to completely penetrate through the tissue.

### BRIEF DESCRIPTION OF THE FIGURES

The accompanying drawings, which are incorporated in and constitute apart of this specification, illustrate embodiments of the invention and, together with the general description of the invention given above, and the detailed description of the embodiments given below, serve to explain various principles of the present invention.

FIG. 1 is a diagrammatical view illustrating the use of one embodiment of a surgical instrument of the present invention inserted through a patient's mouth and esophagus for forming a hole through the abdominal wall;

FIG. 2 is an exploded assembly view of a tissue-penetration instrument embodiment of the present invention and an endoscope;

FIG. 3 is an assembled view of the tissue-penetration instrument of FIG. 2 inserted through a working channel of the endoscope which is shown in cross-section for clarity;

FIG. 4 is an assembled view of the tissue-penetration instrument of FIGS. 2 and 3 with some components thereof shown in cross-section;

FIG. 5 is a partial perspective view of the blunt tip assembly protruding out through the distal end of the hollow support tube;

FIG. 6 is a side view of the blunt tip assembly and hollow support tube depicted in FIG. 5;

FIG. 7 is a partial enlarged view of the blunt tip assembly of FIG. 6;

FIG. 8 is a side view of a blunt tip assembly initially contacting a portion of target tissue;

FIG. 9 is another side view of the blunt tip assembly partially penetrating through the tissue;

FIG. 10 is another side view of the blunt tip assembly completely penetrating through the tissue; and

FIG. 11 is a side view of another tissue penetrating instrument embodiment of the present invention in use with a guide wire.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for penetrating tissue. In general, a tissue-penetrating instrument is provided that has a substantially flexible elongate penetration member with a substantially blunt tissue-penetrating tip at a distal end thereof for penetrating tissue. The instrument can also include a substantially flexible elongate hollow support tube for movably receiving and supporting a portion of the elongate penetration member therein as at least one motion is applied to the distal end of the penetration member. Such hollow support tube may be configured to prevent the elongate penetration member from buckling as axial and/or rotational motions are applied thereto. While the instrument can be used in a variety of applications, it is preferably used in endoscopic or laparoscopic surgery. For example, the instrument can be inserted translumenally through a natural body lumen, and then penetrated through a tissue surface, such as the stomach or colon, to form a puncture hole in the tissue to provide access to other areas of the body, such as the abdominal cavity. The substantially blunt tip of the tissue penetration member is particularly advantageous as it allows the device to penetrate through tissue, while preventing puncture or injury to adjacent tissue, such as organs disposed within the stomach cavity.

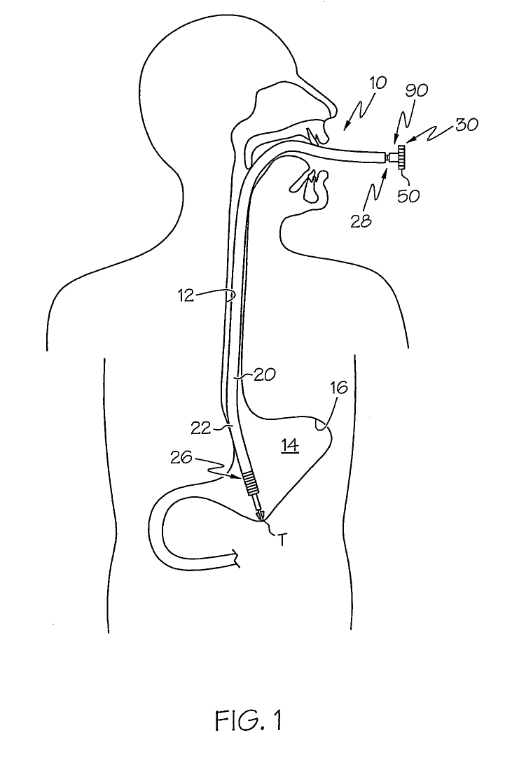

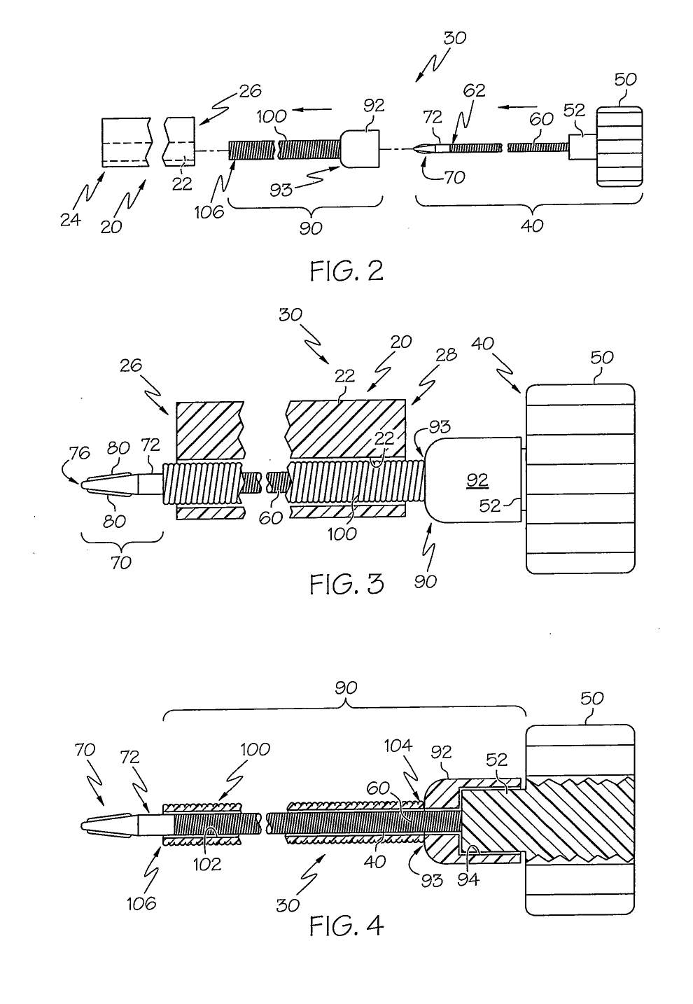

FIG. 1 illustrates, in general form, a tissue-penetration instrument 30 of the present invention that can be used in connection with a conventional endoscope 20 to form an opening through the stomach wall 16. In the example depicted in FIG. 1, the endoscope 20 and tissue-penetration instrument 30 are inserted through the mouth 10 and esophagus 12 into the stomach 14. The endoscope 20 may comprise an elongate flexible tube that may support a camera (not shown) therein. The endoscope 20 may further have one or more working channels 22 (FIGS. 2 and 3) therein that may function as lumens for receiving the tissue-penetration instrument 30 or additional surgical tools therethrough. A variety of different types of endoscopes are known and, therefore, their specific construction and operation will not be discussed in great detail herein.

The endoscope 20 may have a distal end 24 for insertion into the stomach 14 and a proximal end 26 that is accessible to the clinician outside of the patient's mouth or other natural orifice. As used herein, the term “proximal” refers to the portion closest to the clinician and the term “distal” referring to the portion located away from the clinician. It will be further appreciated that, for convenience and clarity, spatial terms such as “vertical”, “horizontal”, “up” and “down” may be used herein with respect to the drawings. However, surgical instruments are used in many orientations and positions, and these terms are not intended to be limiting and/or absolute.

FIGS. 2-7 illustrate, in general form, one exemplary tissue-penetration instrument 30 of the present invention that can be used in connection with a flexible endoscope 20 to form an opening through the wall of an organ. As can be seen in those Figures, the tissue-penetration instrument 30 may comprise a penetration assembly 40 and a support assembly 90. In various embodiments, the penetration assembly 40 comprises a control knob 50 that has a hub portion 52 integrally formed or otherwise attached thereto. The control knob 50 and hub 52 may be fabricated from, for example, a polymeric material such as polycarbonate or other suitable material. Attached to the hub 52 is an elongate penetration member 60 that has a distal end 62. In various embodiments, the elongate penetration member 60 may comprise a hollow flexible tube that may be fabricated from, for example, helically wire wound tubular rope (cable tube), coil pipe, serrated tube, etc. and be attached to the hub 52 by an appropriate adhesive, threads, welds or other mechanical means. In other embodiments, the elongate penetration member 60 may be integrally formed with the hub 52. The distal end 62 of the penetration member 60 is constructed to be inserted translumenally and therefore the penetration member 60 is substantially flexible to allow insertion through a tortuous lumen.

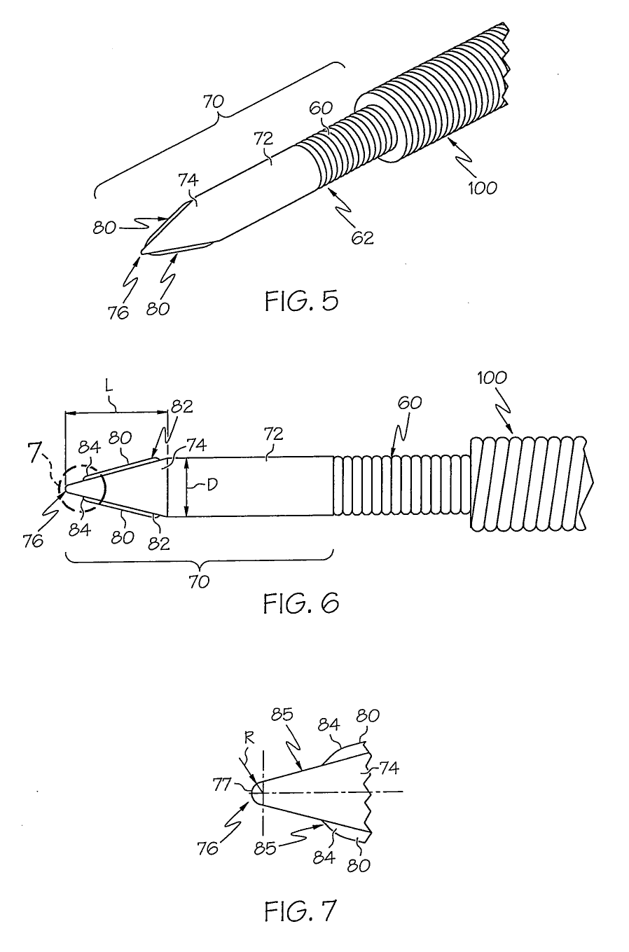

As can be most particularly seen in FIGS. 5-7, a blunt tissue-penetrating tip assembly 70 may be attached to the distal end 62 of the penetration member 60. In various embodiments, the blunt tip assembly 70 may be fabricated from, for example, stainless steel, carbon-filled Nylon or other suitable materials and be attached to the distal end 62 of the penetration member 60 by an appropriate adhesive, threads, welding or other suitable fastening arrangement. FIGS. 5-7 illustrate one form of blunt tissue-penetrating tip assembly 70 that may be employed. As can be seen in those Figures, the blunt tip assembly 70 may have an attachment portion 72 for attachment to the distal end 62 of the penetration member 60 and a substantially conically-shaped body portion 74 that protrudes distally from the attachment portion 72 and terminates in a substantially blunt end 76. In various embodiments, the attachment portion 72 may have the same cross-sectional shape and size as the distal end of the penetration member 60. For example, the attachment portion 72 may have substantially round cross-sectional shape with a diameter “D” of, approximately, 2.8 mm that is substantially identical to the size and cross-sectional shape of the penetration member 60. However, other cross-sectional shapes and sizes could be employed. Also, the substantially conically-shaped body portion 74 may have a length “L” of, for example, 3-5 mm. However, the body portion 74 may have other lengths. The substantially blunt end 76 may be preferably configured to permit proper blunt dissection but may not be sharp enough to cause bleeding under accidental sticks. For example, the blunt end 76 may have a radius “R” of approximately 0.005-0.010 inches (0.127-0.254 mm). See FIG. 7. However, the blunt end 76 may be formed with other radiuses.

As can also be seen in FIGS. 5 and 6, the substantially conically-shaped body portion 74 may also have at least one lateral tine 80 protruding therefrom. In the embodiment depicted in FIGS. 2-10, two lateral tines 80 are employed. In those embodiments, the tines 80 are elongated and have a slightly angled proximal portion 82 and a slightly angled distal portion 84. See FIG. 6. In various embodiments, the end 85 of the distal portion 84 may be approximately 0.25-0.35 mm from the outer surface 77 of the blunt end 76. See FIG. 7. Those of ordinary skill in the art will appreciate that the lateral tines 80 may serve to assist with the tearing of tissue when the blunt tip assembly 70 is rotated into the tissue. Thus, when the blunt tip assembly 70 is rotated, the combination of the compression into the tissue and the rotation of the lateral tines 80 causes the dissection of the tissue. It will be further appreciated that other shapes and numbers of tine configurations could be employed. For example, a single helically wound tine could be employed or multiple segmented tines, etc. could be employed. In still other embodiments, no lateral tines may be provided on the body portion 74.

Various embodiments of the tissue-penetration instrument 30 may also include a flexible support assembly 90. As can be seen in FIGS. 2-4, for example, the support assembly 90 may comprise a support knob 92 that is sized to be rotatably and axially received on the hub 52. In particular, as can be seen in FIG. 4, the support knob 92 may have a cavity 94 therein sized to enable the support knob 92 to be rotated and axially slid on the hub 52. The support knob 92 may have a distal end 93 to which a flexible hollow support tube 100 is attached. The flexible hollow support tube 100 may comprise, for example, a commercially available “coil” tube that has a substantially smooth interior surface 102. See FIG. 4. The support tube 100 may have a proximal end 104 that is attached to the distal end 93 of the support knob 92 by, for example, an appropriate adhesive, welding, etc. such that when the support knob 92 is inserted onto the knob hub 52 as shown in FIG. 3, the penetration member 60 extends through the support tube 100. The support tube 100 may be sized relative to the penetration member 60 such that the elongate penetration member 60 may freely rotate and move axially therein while providing support thereto to prevent buckling of the penetration member 60 when the penetration member 60 is placed under load as will be further discussed below. The distal end 106 of the support tube 100 is constructed to be inserted translumenally and therefore the support tube 100 is substantially flexible to allow insertion through a tortuous lumen.

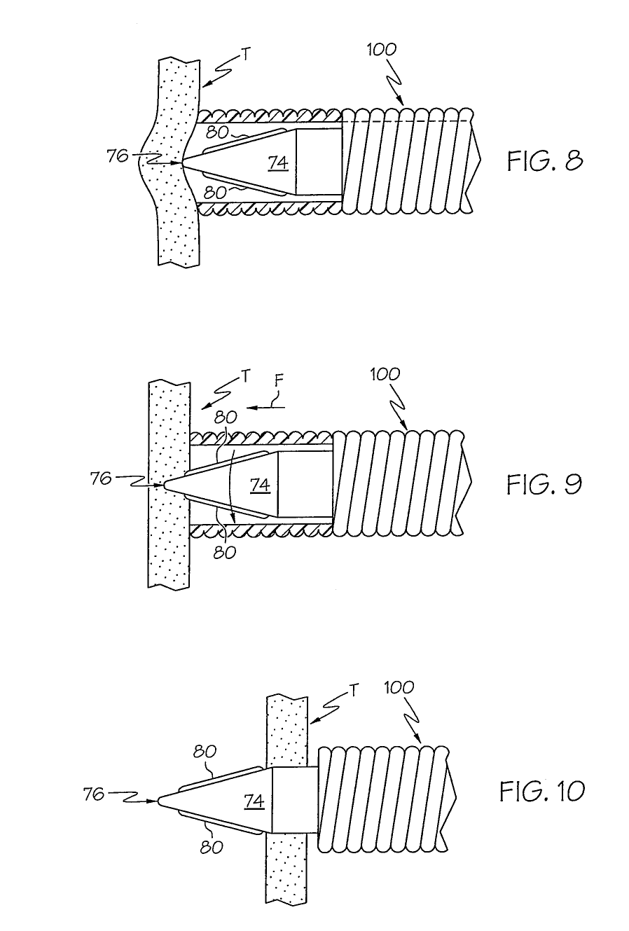

Use of the tissue-penetration instrument 30 will now be described with reference to FIGS. 1 and 8-10. As can be seen in FIG. 1, the procedure is commenced by inserting the endoscope 20 through the patient's natural orifice 10 (mouth) and esophagus 12 into the stomach 14. The camera (not shown) within the endoscope 20 may be used to locate the distal end of the endoscope in the desired location. Thereafter, the support assembly 90 may be inserted through a working channel 22 in the endoscope 20. The penetration member 60 may be located within the support tube 100 during insertion and positioning of the support tube 100 such that the blunt tip assembly 70 is withdrawn into the support tube 100 to prevent any accidental contact between the blunt end 76 and non-target tissue and/or organs during the positioning process. Once the support tube 100 and penetration member 60 have been located in the desired position, the surgeon may grasp the control knob 50 and apply an axial force thereto to cause the blunt tip assembly 70 to protrude out of the distal end 106 of the support tube 100 and contact the target tissue “T”. See FIG. 8. Once the blunt end 76 has contacted the target tissue T, the surgeon may then apply a twisting or rotating motion to the control knob 50 while pushing the control knob 50 forward (arrow “F” in FIG. 9) until the blunt end 76 pushes through the target tissue T. See FIG. 10. In the event that the target tissue T is difficult to initially penetrate with the blunt end 76, the surgeon may create an initial cut or partial penetration in the target tissue T by inserting a conventional knife or needle knife (“dissection device”) through a working channel in the endoscope. In doing so, the surgeon may not create a defect in the target tissue T that would extend completely through the tissue T. The blunt end 76 is then pushed into the partial penetration as the control knob 50 is rotated. Rotation of the control knob 50 causes the tip assembly 70 to rotate. If employed, the lateral tines 80 may help to tear the tissue T as the tip assembly 70 is rotated. The combination of the compression on the tip assembly 70 and the action of the lateral tines 80 causes the tip assembly 70 to penetrate the tissue T. After the tip assembly 70 has completely penetrated the target tissue T, the distal end of the endoscope 20 may be inserted through the tissue wall. Thereafter the tissue-penetration instrument 30 may be removed from the endoscope 20 to permit other surgical instruments to be inserted through the endoscope's working channel to complete the surgical procedure.

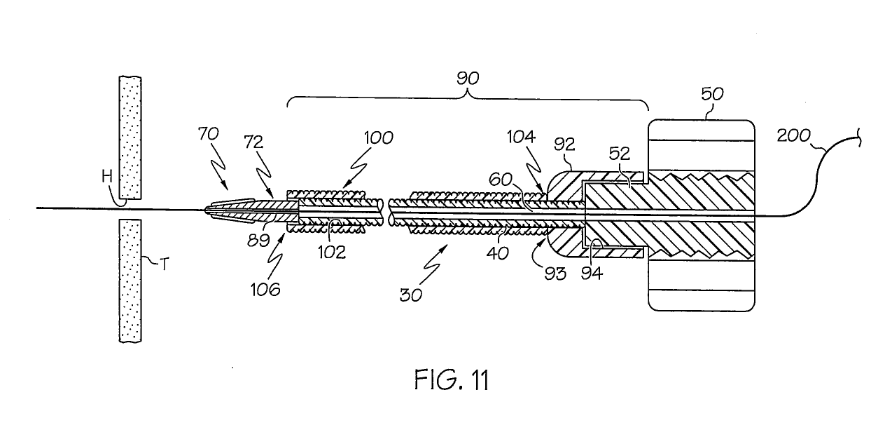

In various alternative embodiments, this process may be aided by placing a guide wire 200 through an axial hole 89 in the blunt tip assembly 70. See FIG. 11. When employing such embodiment, after the guide wire 200 has been inserted through the hole “H” formed in the tissue T, the tissue penetrating instrument 30 may then be removed to permit a conventional dilating balloon (not shown) to be inserted over the guide wire 200 to enable the dilating balloon to be positioned within the hole “H” formed in the tissue T and inflated to enlarge the hole “H” in a known manner.

Those of ordinary skill in the art will appreciate that the present invention represents a vast improvement over prior tissue penetration devices and methods. In particular, prior dissection devices, such as trocars and the like are ill-suited for use in natural orifice translumenal endoscopic surgery due to the rigid nature of their cannulas. Other devices, however, that have been used in the past that can be inserted through the working channel of an endoscope and used to form an opening in a wall of tissue are generally sharp and can lead to undesirable sticks and cuts in surrounding tissue and organs particularly when the device penetrates through the wall of target tissue. The various embodiments of the present invention, however, may avoid those shortcomings.

While several embodiments of the invention have been described, it should be apparent, however, that various modifications, alterations and adaptations to those embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The instruments disclosed herein can be designed to be disposed of after a single use, or they can be designed to be used multiple times. In either case, however, the device can be reconditioned for reuse after at least one use. Reconditioning can include an combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Those of ordinary skill in the art will appreciate that the devices disclosed herein may also be provided in kit form. For example, a kit may include a tissue penetration instrument 30 of the present invention in combination with a disposable endoscope that has at least one working channel therein

The invention which is intended to be protected is not to be construed as limited to the particular embodiments disclosed. The embodiments are therefore to be regarded as illustrative rather than restrictive. Variations and changes may be made by others without departing from the spirit of the present invention. Accordingly, it is expressly intended that all such equivalents, variations and changes which fall within the spirit and scope of the present invention as defined in the claims be embraced thereby.

1. A tissue-penetration instrument comprising:
a substantially flexible elongate penetration member having a proximal end and a distal end, said distal end having a substantially blunt tissue penetrating tip thereon;
an axial passage through said substantially flexible elongate penetration member and said substantially blunt tissue penetrating tip thereof for accommodating at least one guide member therethrough; and
an elongate hollow support tube having a passage extending therethrough sized to movably support a portion of said substantially flexible elongate penetration member therein and prevent buckling thereof as at least one motion is applied to said distal end of said substantially flexible elongate penetration member, said elongate hollow support tube sized to extend through a working channel of an endo scope.

2. The tissue-penetration instrument of claim 1 wherein said substantially blunt tissue penetrating tip comprises an attachment portion for attachment to said distal end of said substantially flexible elongate penetration member and a substantially conically-shaped body portion protruding distally from said attachment portion and terminating in a substantially blunt end.

3. The tissue-penetration instrument of claim 2 further comprising at least one lateral tine on said substantially blunt tissue penetrating tip.

4. The tissue-penetration instrument of claim 2 wherein said substantially blunt end has a radius of 0.005 to 0.010 inches.

5. The tissue-penetration instrument of claim 1 further comprising a control knob formed on said proximal end of said substantially flexible elongate penetration member.

6. The tissue-penetration instrument of claim 5 further comprising a support knob on a proximal end of said elongate hollow support tube, said support knob movably supported on a portion of said control knob.

7. The tissue-penetration instrument of claim 6 wherein said support knob is configured to be selectively axially and rotatably movable relative to said control knob.

8. The tissue-penetration instrument of claim 7 wherein said control knob has a hub portion thereon and wherein said support knob is axially and rotatably movably supported on said hub portion.

9. The tissue-penetration instrument of claim 1 wherein said elongate hollow support tube comprises a coil tube.

10. A method for processing a tissue-penetration instrument for surgery, the method comprising:
obtaining the tissue-penetration instrument of claim 1;
sterilizing the tissue-penetration instrument; and
storing the tissue-penetration instrument in a sterile container.

11. (canceled)

12. The tissue-penetration instrument of claim 1 wherein said at least one guide member comprises a guide wire.

13. A tissue-penetration instrument comprising:
a substantially flexible elongate penetration member having a proximal end and a distal end;
a control knob on said proximal end of said substantially flexible elongate penetration member to enable axial and rotational motions to be applied thereto;
a blunt tissue-penetrating tip assembly coupled to said distal end of said substantially flexible elongate penetration member, wherein said substantially flexible elongate penetration member, said control knob, and said blunt tissue-penetrating tip assembly each have an opening therethrough to define an axial passage for accommodating at least one guide member therethrough; and
an elongate hollow support tube having a passage extending therethrough sized to movably support a portion of said substantially flexible elongate penetration member therein and prevent buckling thereof as at least one of said axial and rotational motions is applied to said control knob.

14. The tissue-penetration instrument of claim 13 further comprising at least one lateral tine formed on said blunt tissue-penetrating tip assembly.

15. The tissue-penetration instrument of claim 13 wherein said blunt tissue-penetrating tip assembly is coupled to said distal end of said elongate penetration member by a fastener medium selected from the group of fastener mediums consisting of adhesives, threads, and welds.

16. A method for penetrating tissue, comprising:
inserting the tissue penetration instrument of claim 13 through a body lumen; and
applying an axial motion to the elongate penetration member to cause the substantially blunt tip thereon to penetrate through the tissue.

17. The method of claim 16 further comprising applying a rotational motion to the elongate penetration member during said applying an axial motion.

18. The method of claim 16 wherein said inserting comprises:
inserting an endoscope having at least one working channel therein through the body lumen; and
inserting a portion of the tissue penetration instrument through one of the working channels in the endoscope.

19. The method of claim 16 wherein the blunt tissue-penetrating tip of the elongate tissue-penetration member is completely received within the elongate hollow support tube during said inserting.

20. The method of claim 16 wherein said inserting comprises:
inserting the elongate hollow support tube through the body lumen; and
inserting the tissue-penetration member through the hollow support tube.

21. The method of claim 20 wherein said inserting the elongate hollow support tube through the body lumen comprises:
inserting an endoscope having at least one working channel therein through the body lumen; and
inserting the elongate hollow support tube into one of the working channels in the endoscope.

22. A method for penetrating tissue comprising:
inserting a substantially flexible dissection device through a body lumen;
piercing partially through a portion of tissue with the dissection device to form a partial penetration in the tissue;
inserting a substantially flexible tissue-penetration member having a substantially blunt tissue penetrating tip through the body lumen such that the blunt tissue penetrating tip is positioned within the partial penetration formed in the tissue; and
applying at least one motion to the tissue-penetration member to cause the blunt tissue penetration tip to completely penetrate through the tissue.

23. The method of claim 22 wherein said inserting a substantially flexible dissection device comprises:
inserting an endoscope having at least one working channel therein through the body lumen; and
inserting the substantially flexible dissection device through one of the working channels in the endoscope and wherein said inserting a substantially flexible tissue-penetration member comprises:
removing the substantially flexible dissection device from the working channel; and
inserting the substantially flexible tissue-penetrating member through the working channel.

24. The method of claim 22 further comprising:
inserting a guide member through the substantially flexible tissue-penetration member such that the guide member extends through a hole formed through the tissue by the blunt tissue penetration tip; and
removing the substantially flexible tissue penetration member from the body lumen while permitting the guide member to extend through the hole formed in the tissue.

25. A surgical kit comprising:
an endoscope; and
a tissue-penetration instrument of claim 1.
